(19) United States
(12) Patent Application Publication
(10) Pub. No.: US 2019/0352719 A1
(43) Pub. Date: Nov. 21, 2019

(54) CALCIUM FLUORESCENT PROBES TO ASSESS ORAL CARE COMPOSITION EFFICACY IN A BIOFILM

(71) Applicant: The Procter & Gamble Company, Cincinnati, OH (US)

(72) Inventors: Yunming SHI, Beijing (CN); Swapna BASA, Beijing (CN); Ross STRAND, Singapore (SG)

(21) Appl. No.: 16/530,459

(22) Filed: Aug. 2, 2019

Related U.S. Application Data
(63) Continuation of application No. PCT/CN2017/075538, filed on Mar. 3, 2017.

(51) Int. Cl. C12Q 1/6883; C12Q 1/02; C12Q 1/18; G01N 33/52; G01N 33/58; G01N 33/84

(52) U.S. Cl. CPC C12Q 1/6883; C12Q 1/025; G01N 33/84; G01N 33/52; G01N 33/582; C12Q 1/18

(57) ABSTRACT

A method of quantitating calcium in a biofilm is an effective way of assessing an oral care composition's efficacy to help inhibit biofilm formation or help disrupt biofilm.

### FIELD OF THE INVENTION

The present disclosure is directed to methods for assessing an care composition's efficacy to help inhibit biofilm formation or help disrupt biofilm.

### BACKGROUND OF THE INVENTION

Methods for quantitating the efficacy of oral care compositions (e.g., toothpaste, mouthwash etc.) at dislodging cells from biofilm test surfaces or inhibiting or delaying the accumulation of cells on a test surface have generally been described. Also, fluorescent probes, and confocal laser scanning microscopy (CLSM), have been generally used in studying biofilm. Dental plaque is an example of bacterial biofilm. Dental plaque forms tartar and is associated with oral diseases such as caries and periodontal disease (e.g., gingivitis and periodontitis). Dental plaque can give rise to dental caries caused by the acid from the bacterial degradation of fermentable sugar. Therefore, dental plaque control and removal is important and the objective of many oral care products and regimens. Therefore, there is generally a continuing need to further understand the mechanisms of dental plaque formation and design oral care compositions that control or remove dental plaque.

One such mechanism of dental plaque formation is the role of calcium ions. Bacteria and biofilms are negatively charged, as are teeth. Thus theoretically, bacteria should not stick to the teeth given the repulsion of negative charges. However, positively charged calcium ions in the saliva mask these negative charges thereby allowing the bacteria to come in close contact with the tooth surface. Then, stronger attractive surface forces that work at very small distances (e.g., Van der Waals forces) become more influential allowing the bacteria to attach and multiply on the tooth surface contributing to the formation of dental plaque. In the plaque, bacteria will continue to multiply, secreting acid from degradation of sugar, and concentrating calcium in a positive feedback loop of plaque formation. The amount of calcium in plaque is two to three times greater than in saliva.

One solution to inhibit dental plaque formation is the use of calcium binders in oral care compositions. These calcium binders are used to displace the harmful calcium from the bacterial biofilm because the binder has a stronger binding force for calcium than those forces that bind the calcium in the biofilm. Oral care compositions can be better designed to displace these calcium ions so as to help disrupt or reduce the biofilm. The challenge is the displacement of calcium ions from within the biofilm without demineralizing the tooth though removal of calcium ions from the tooth structure. Another approach is the use of an abrasive. For example, the abrasive may be included as part of a toothpaste and through the tooth brushing action with the toothpaste, the dental plaque can be physically disrupted. Yet another approach is the use of an antimicrobial agent to inhibit the presence of bacteria that may contribute to the formation of dental plaque. Nevertheless, there remains a continuing need for improved actives or improved formulations for actives delivery, or improved oral care compositions that generally inhibit or disrupt biofilm formations. Accordingly, there is a need for methods at quantitating calcium in a biofilm as to assess the efficacy of oral care compositions to inhibit biofilm formation or to disrupt biofilm.

### SUMMARY OF THE INVENTION

The present invention addresses at least one of these needs by providing a method of quantitating calcium in a biofilm comprising the steps: optionally treating, preferably treating, the biofilm with an oral care compositions; labeling the optionally treated biofilm with a biofilm fluorescent probe; labeling the optionally treated biofilm with a calcium fluorescent probe; and quantitating the labeled cells by measuring fluorescence light emitted from the labeled cells by, for example, confocal laser scanning microscopy. Preferably the biofilm fluorescent probe is selected from a microbial fluorescent probe, an extracellular polymeric substances (“EPS”) fluorescent probe, or combination thereof. Preferably the oral care composition comprises a calcium binder (e.g., calcium chelator).

Another aspect of the invention provides a kit comprising: a biofilm fluorescent probe; a calcium fluorescent probe; and optionally use instructions for use in biofilm.

The present invention is based, in part, upon the surprising discovery that calcium probes can be used in quantitating calcium ions in biofilm.

One advantage of the present method is that the calcium and microbial/EPS probes fluoresce at different excitation/emission wavelength and as such fluorescence intensity and co-localization of the probes in bacteria of biofilm can be determined.

Another advantage of the present invention is the methods can be used to identify more efficacious oral care compositions to displace calcium in biofilm or in biofilm formation.

Yet another advantage of the present invention is allowing the study of calcium at varying depths of the dental biofilm without damaging natural structure of biofilm.

Yet another advantage of the present invention is the methods can be used to demonstrate to consumers and dental professionals how oral compositions displace calcium in biofilm or in biofilm formation.

These and other features, aspects and advantages of specific embodiments will become evident to those skilled in the art from a reading of the present disclosure.

### BRIEF DESCRIPTION OF THE DRAWINGS

The embodiments set forth in the drawings are illustrative in nature and not intended to limit the invention defined by the claims. The following detailed description of the illustrative embodiments can be understood when read in conjunction with the following drawings, where like structure is indicated with like reference numerals and in which:

FIG. 1 is a perspective view of an oral splint with hydroxyapatite disks attached thereto;

FIG. 2 is a perspective view of the hydroxyapatite disk having grooves therein;

FIG. 3 is a schematic of a cross sectional view of the groove with biofilm therein;

### DETAILED DESCRIPTION OF THE INVENTION

The following text sets forth a broad description of numerous different embodiments of the present disclosure. The description is to be construed as exemplary only and does not describe every possible embodiment since describing every possible embodiment would be impractical, if not impossible. It will be understood that any feature, characteristic, component, composition, ingredient, product, step or methodology described herein can be deleted, combined with or substituted for, in whole or part, any other feature, characteristic, component, composition, ingredient, product, step or methodology described herein. Numerous alternative embodiments could be implemented, using either current technology or technology developed after the filing date of this patent, which would still fall within the scope of the claims.

One aspect of the present disclosure is directed to method of quantitating calcium of a biofilm comprising the steps: optionally treating, preferably treating, the biofilm with an oral care compositions; labeling the optionally treated biofilm with a biofilm fluorescent probe; labeling the optionally treated biofilm with a calcium fluorescent probe; quantitating the labeled biofilm by measuring fluorescence light emitted from the labeled biofilm and quantitating calcium ions by measuring fluorescence light emitted from the labeled calcium ions within the defined biofilm area by, for example, confocal laser scanning microscopy (CLSM). These steps need not be conducted in any specific order.

Treating the Biofilm with an Oral Care Composition

The term “biofilm” refers to the layer(s) of cells attached to a surface. A biofilm can a bacterial biofilm that includes both alive and growing microbe cells as well as dead microbe cells. The biofilm can be composed of one cell type or it may be composed of two or more cell types, for example, a biofilm complex that is a multispecies bacterial community. A specific type of biofilm is “dental biofilm” (also known as “plaque biofilm,” used herein interchangeably) which is biofilm that typically forms on tooth surfaces in the human mouth). Bacteria in a plaque biofilm have significantly different physiological characteristics, e.g. increased resistance to detergents and antibiotics, making biofilm research highly important. A non-limiting list of oral bacterial species is described at U.S. Pat. No. 6,309,835 B1, column 7, lines 12-30. These adherent microbe cells are frequently embedded within a self-produced matrix of extracellular polymeric substance (EPS). EPS are biopolymers of microbial origin in which biofilm microorganisms are embedded. J. Bacteriol. 2007, 189(22):7945. Biofilm extracellular polymeric substance is a polymeric conglomeration generally composed of calcium, extracellular DNA, proteins, and polysaccharides. The biofilm may be either in vitro biofilm or in situ biofilm. Preferably the biofilm is in situ plaque biofilm because it more accurately reflects the conditions of the human mouth by providing a natural and undistributed biofilm. One approach that lends itself well to quantitating calcium in the biofilm over a defined period of time is using in situ plaque biofilm.

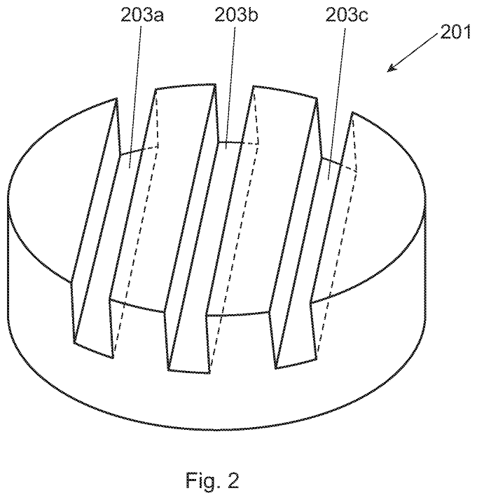

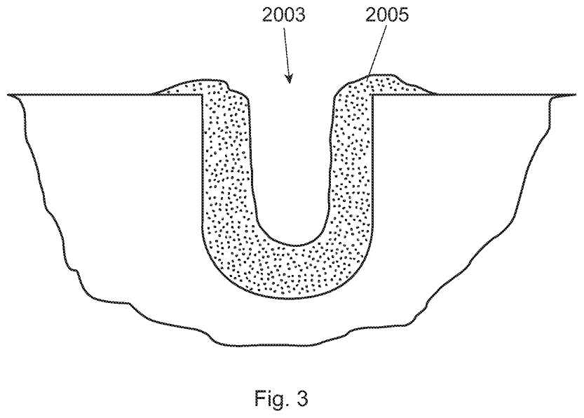

A number of different surfaces for which the biofilm may attach are contemplated. These surfaces may include, for example, human enamel, bovine enamel, bovine dentine, hydroxyapatite, polished glass, and titanium. Considering the roughness of the surface of the substrate and its free energy are important factors for the in situ growth of plaque biofilm. Enamel or hydroxyapatite are preferred surfaces to mimic a natural substrate for growth of plaque biofilm. On the other hand, due auto-fluorescence of enamel, hydroxyapatite is more preferred for the in situ growth of plaque biofilm. Hydroxyapatite, also called hydroxylapatite, (“HA”) is a mineral form of calcium apatite generally having the formula Ca₁₀(PO₄)₆(OH)₂. In a particularly preferred approach, HA containing pieces (e.g., small disks) are used. These HA pieces are relatively small, preferably having an overall volume of 7 mm3 to 110 mm3, preferably from 25 mm³ to 35 mm³. The HA pieces are designed having a plurality of grooves (to allow plaque biofilm to attach inside the groove). For clarification, either in situ or in vitro plaque biofilm may be used to attach to the inside of the groove(s), but in situ plaque biofilm is preferred. The plurality of grooves preferably have dimensions that are from 50 um to 500 um deep and from 50 um to 500 um wide, more preferably from 100 um to 400 um deep and from 100 um to 400 um wide, even more preferably at least one of the grooves is from 250 um to 350 um deep and from 250 um to 350 um wide. Without wishing to be bound by theory, many human subjects do not care to have an oral appliance (containing these HA pieces) for more than two to three days. With grooves smaller than these dimensions, the groove is filled up with in situ plaque biofilm thereby not allowing the subject oral care composition and/or fluorescent probes to penetrate into the groove. On the other hand, if the dimensions of the groove are too large then the grooves do not lend themselves well to biofilm growth or attachment, particularly if the human subject is only going to wear the oral appliance for two to three days. In addition, these preferred groove dimensions provide for an optimum cross section view by conventional CLSM. In a specific example, and turning to FIG. 2, the HA disk (201) has three parallel grooves (203) (the two sides' grooves (203a and 203c) are 300 um wide and 300 um deep; while the middle grove (203b) (in between the two side grooves) is 500 um wide and 500 um deep). The middle groove is designed wider and deeper than the two sides' grooves so that the HA disk can be more easily separated into two identical half-disks for head-to-head comparison purposes. FIG. 3 is a schematic of a cross sectional view of the groove (2003) with biofilm (2005) therein.

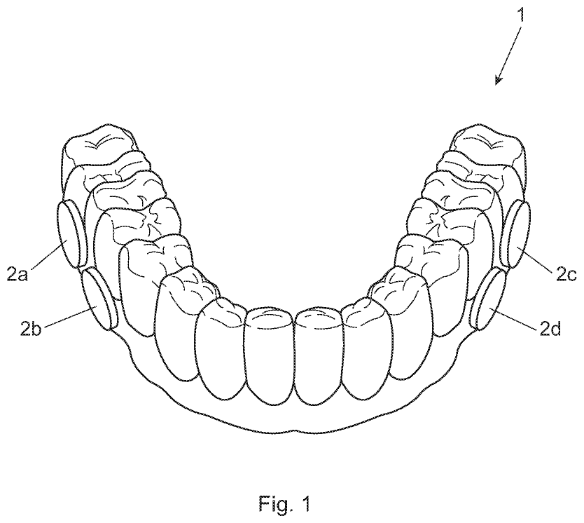

Preferably the in situ plaque biofilm is attached to the surface of HA pieces as a result of the HA pieces being attached to an oral appliance (e.g., oral splint or mouthpiece) worn by human subjects for a defined period of time. This defined period of time is preferably from 6 hours to 4 days, more preferably from 1 day to 3 days, alternatively about 2 days. Accordingly, the method may comprise the step of having human subjects wearing the oral appliance for 6 hours to 4 days, preferably 1-3 days, more preferably 2 days; wherein at least a portion of the oral appliance comprises HA as a surface of the biofilm, and wherein the biofilm is an in situ plaque biofilm. The term “oral appliance” means a device that can be temporarily worn inside the oral cavity (i.e., mouth) of a human subject for up to multiple days at a time (but temporarily removed during eating or oral hygiene and the like). Non-limiting examples of an oral appliance include an oral splint, mouthpiece, and retainer. The oral appliance preferably has a plurality of HA containing pieces (e.g., small disks) releasably attached thereto. In other words, the human subject wears the oral appliance as to allow biofilm to attach/grow to the surfaces and grooves of the HA disk. After 6 hours to 4 days, preferably 2-3 days, more preferably 2 days, the HA disks are removed by the oral appliance that was worn by the human subject. FIG. 1 is an example of a splint (1) having a plurality of HA disks (2a, 2b, 2c, 2d) releasably attached to the splint. The splint (1) is worn over the teeth of a human subject (not shown) for a defined period of time with the objective of having biofilm grow/attach to the HA disks, preferably in grooves of the HA disks. In FIG. 1, the plurality of HA disks are on the interdental buccal side of the oral applicant. Although not shown in FIG. 1, a preferred location of the HA pieces is on the lingual side of the appliance. Without wishing to be bound by theory, the lingual side is even more difficult to brush thereby providing in situ plaque biofilm that is likely thicker (i.e., grows or forms more quickly than from other locations in the oral cavity). Moreover, there is also a suggestion that the in situ plaque biofilm resulting from the lingual side maybe by more toxic or pathogenic.

The biofilm may be treated with the oral care composition either in vivo or ex vivo. “In vivo” means that which takes place within the organism, specifically within the oral cavity of the human subject. For example, the human subject may wear an oral splint (and the HA disks releasably attached thereto) while using the oral care composition. “Ex vivo” means that which takes place outside an organism, specifically outside the oral cavity of the human subject. For example, after the splint is worn, the HA disks may be removed and then treated with the subject oral care composition. Such an ex vivo approach is preferable when quantitating calcium in the biofilm or quantitating the calcium retention in the biofilm (e.g., single or multiple oral care product/composition usage).

The oral care composition may be any composition that is designed to be primarily used for oral hygiene in humans. The term “oral care composition” can be a single ingredient (e.g., a calcium binder) or a formulation with multiple ingredients. The oral care composition may vary not only in ingredients but also in the concentration of these ingredients. Preferably these ingredient(s) are safe for use in the oral cavity of humans. Non-limiting examples of oral care compositions may include dentifrice, toothpaste, mouthwash, leave-on gel, gel, etc., or combinations thereof.

Oral care compositions preferably comprise a calcium binder to: (i) prevent or mitigate dental biofilm formation; (ii) disrupt existing dental biofilms; (iii) prevent or mitigate further biofilm growth; or (v) combinations thereof. The oral care compositions may comprise more than one calcium binder. A calcium binder displaces calcium by way of chelating, binding, removing, precipitating, or otherwise. One example of a calcium binder is sodium bicarbonate (i.e., baking soda) or ethylenediaminetetraacetic acid (EDTA). Such oral care composition may typically contain from 0.0025% to 75%, by weight of the composition, of the ion binder. The oral care product may comprise additionally an abrasive to help physically disrupt the dental biofilm. An example is an abrasive that in toothpaste is silicate or sodium carbonate. The oral care product may additionally comprise an antimicrobial agent that help mitigate the growth or presence of bacteria that contribute to dental biofilm formation. An example of an antimicrobial agent is zinc or a zinc salt (e.g., zinc chloride). The oral care product may additional comprise both an abrasive and an antimicrobial agent.

In an alternative embodiment, the biofilm attaches to a test piece of mammalian (e.g., human or bovine) enamel surface. That is, pieces of enamel are subject to a relatively longer term study (e.g., 5-21 days). These pieces can also be releasably attached to an oral care appliance and worn by a human subject. This in situ method can be used to assess the effect of an oral care composition on: calcium levels in the biofilm and/or biofilm formation and/or biofilm disruption.

The method may comprise treating the biofilm with the oral care composition for a treatment contact time from: 1, 3, 5, 10, 30, or 45 seconds; or 1, 2, 3, 4, or 5 minutes; or 5, 10, 30, 60, 120 minutes; or 1 to 2 days; or 3 seconds to 48 hours; preferably from 1 minute to 3 minutes; or combinations thereof.

Labeling the Biofilm with a Microbial and/or EPS Fluorescent Probe

The biofilm is labeled with a biofilm fluorescent probe. Preferably the biofilm fluorescent probe is selected from a microbial fluorescent probe, an extracellular polymeric substances (“EPS”) fluorescent probe, or combination thereof. “Microbial fluorescent probe” means a fluorescent probe that binds to microbes of a biofilm and emit fluorescence at a certain wavelength. One class of such probes includes fluorescently labeled oligonucleotides, preferably rRNA-directed oligonucleotides. Non-limiting examples include SYTO™ branded dyes. One specific example is SYTO® 9 Green Fluorescent Nucleic Acid Stain, wherein excitation is a 485 (DNA) and 486 (RNA), and light emission is detected at 498 (DNA) and 501 (RNA). And another specific example is SYTO® 40 Blue Fluorescent Nucleic Acid Stain, wherein excitation is a 420 (DNA), and light emission is detected at 441 (DNA). A benefit of using this stain with calcium fluorescent probe is the different light emission wavelength allows these to be used concurrently in the same biofilm sample. Also within this class of rRNA-directed oligonucleotides dyes, a sub-class of dyes may be used to distinguish between dead or alive microbes. That is, the microbial fluorescent probe may comprise a first probe specific for living bacteria and a second probe specific for dead bacteria. “Extracellular polymer substances (“EPS”) fluorescent probe” means a fluorescent probe that binds to extracellular polymeric substances of a biofilm and emit fluorescence at a certain wavelength. One class of EPS fluorescent probes includes a fluorescently labeled lectin. The term “fluorescently labeled lectin” is also inclusive of lectin derivatives. One advantage of the use of EPS fluorescent probes is avoiding the need to purify EPS from the other components of the biofilm to understand the effect of oral care composition upon calcium ion contained in the EPS portion of the biofilm. Indeed, it has been reported that it is often difficult to purify EPS matrix constituent apart from other components of the biofilm such as cells. J. Bacteriol. 2007, 189(22):7945. A commercially available example of a microbial fluorescent probe is Molecular Probes' LIVE/DEAD® BacLight™; and an EPS fluorescent probe is Molecular Probes™ Concanavalin A™, Alexa Fluor® 594 Conjugate fluorescence stains. See also U.S. Pat. No. 6,309,835 B1, at column 8, Table 1. One approach in utilizing the microbial fluorescent probes is to assess the effectiveness of abrasive and/or antimicrobials on dental biofilm. These microbial and/or EPS fluorescent probes are widely available as well as the procedure details in how to use them to quantitatively determine the amount of microbes and/or EPS as well as quantitatively determine what portion of these microbes are alive or dead.

In one example, the biofilm fluorescent probe is the microbial fluorescent probe, wherein the method further comprises the step of defining a microbial area of the biofilm by measuring fluorescent light emitted from the labeled microbial biofilm. In other words, the method is able to localize or spatially define the microbes within the biofilm. This measurement can be done with or without regard to fluorescence intensity. One preferred instrument in performing such measurements is confocal laser scanning microscopy (CLSM). As discussed in further detail below, preferably the method further comprises the step of quantitating the labeled calcium within the microbial defined area of the biofilm (i.e., co-localization)

In another example, the biofilm fluorescent probe is the EPS fluorescent probe, wherein the method further comprises the step of defining an EPS area of the biofilm by measuring fluorescent light emitted from the labeled EPS biofilm. In other words, the method is able to localize or spatially define the EPS within the biofilm. This measurement can be done with or without regard to fluorescence intensity. One preferred instrument in performing such measurements is confocal laser scanning microscopy (CLSM). As discussed in further detail below, preferably the method further comprises the step of quantitating the labeled calcium within the EPS defined area of the biofilm (i.e., co-localization).

In yet another example, the method utilizes the combination of microbial fluorescent probes and EPS fluorescent probes.

The subject biofilm is generally incubated with the microbial and/or EPS probe in dark for 15-60 minutes, preferably 30 minutes and excitation light is provided to the incubated biofilm at a wavelength according to instruction manuals of the microbial and/or EPS probes or relevant literature/patent references. The wavelength of light emission detection as well as the procedure details in how to use microbial and/or EPS probes are determined according to these manuals and references.

Labeling the Biofilm with a Calcium Fluorescent Probe

The biofilm is labeled with a calcium fluorescent probe. Examples of a calcium fluorescent probe suitable for labeling the biofilm may be any one or more of the following compounds:

(a) Fluo-3™, AM™, cell permeant fluorescence stains;
(b) Fluo-3™, Pentapotassium Salt, cell impermeant fluorescence stains;
(c) Fluo-4™, AM™, cell permeant fluorescence stains;
(d) Fluo-4™, Pentapotassium Salt, cell impermeant fluorescence stains;

### (e) Fluo-4 Direct™ Calcium Assay Kit;

(f) Mag-Fluo-4™, Tetrapotassium Salt, cell impermeant fluorescence stains; and
(g) Fluo-5F™, AM™, cell permeant fluorescence stains.
One or more of these probes may be available from ThermoFisher Scientific Company, Waltham, Mass.

The subject biofilm is generally incubated with the calcium probe in dark for 15-60 minutes, preferably 30 minutes, and excitation light is provided to the incubated biofilm at a wavelength according to instruction manuals of calcium probes or relevant literature/patent references. The wavelength of light emission detection as well as the procedure details in how to use calcium probes are determined according to these manuals and references.

### Quantitating the Labeled Calcium or Labeled Biofilm by Measuring Emitted Fluorescence Light

The method of the present invention comprises the step of quantitating the labeled calcium, and optionally the labeled biofilm, by measuring fluorescence light emitted from the respective fluorescent probe(s). Quantitating may also include assessing the intensity of fluorescence in a defined area of the biofilm. One preferred instrument in performing such quantification is confocal laser scanning microscopy (CLSM). Commercially available software is able to quantify fluorescence of the pixels from images taken. Three dimensional images can be constructed from a number of single images taken of the labeled calcium/biofilm.

### EXAMPLES

Data is provided on the fluorescence intensity ratio of Calcium/EPS in biofilm and biofilm thickness for two sodium bicarbonate containing solutions, one commercially available sodium bicarbonate containing toothpaste and a negative control. Methodology is first described.

The substrate for biofilm growth is described. Hydroxyapatite (“HA”) disks are used for in situ growth of biofilm. The HA disks are designed having three parallel grooves (300 um wide, 300 um deep for two sides' grooves, while 500 um wide, 500 um deep for the middle groove) in each disk. When attaching disks to subject's mouth, keeping these grooves vertical, to mimic interproximal gap between teeth, the hard-to-clean area where plaque accumulates, this model allows the collection of undisturbed natural grown plaque biofilm from the grooves. HA disks are manufactured by Shanghai Bei'erkang biomedicine limited company.

Human subjects wearing a splint are described. Each subject wears up to 12 HA disks on the splint to make sure at least 9 HA disks are available after 48 hours. A non-limiting example of such a splint and HA disks are shown in FIG. 1. The device (1) holds a plurality of HA disks (2a-2d). Although not shown in FIG. 1, the disks can be positioned such that the recede in the inter-dental space between the teeth (since this location is prone to plaque (given the difficulty in cleaning etc.)). The subjects withdraw the splint (the splint stored in an opaque container under humid conditions) only during meals and to perform oral hygiene procedures Immediately thereafter, the splint is worn again. Subjects are asked to use a straw when drinking.

The procedure for in situ biofilm release from HA disk is described. All HA disks are removed from the splint at 48 hours by tweezers. Tweezers are used to hold the edge of HA disks and transfer the HA disk to a 2 ml centrifuge tube containing PBS (phosphate buffered saline) solution. Tweezers are washed thoroughly (water; 75% alcohol; and then deionized water) before every disk transfer.

The preparation for PBS solution is described. One phosphate buffer saline tablet (available from Sigma-Aldrich Corp., MO, USA) is added to 200 grams deionized water in a 250 ml beaker. After stirring thoroughly, the solution is stored at 4° C. for up to 30 days before usage.

The preparation for two sodium bicarbonate solutions is described. 20 and 60 grams of sodium bicarbonate (Lot#KBAKR08, available from Beijing InnoChem Science & Technology Co., Ltd.) is separately added to deionized water to a final weight of 100 grams in a 200 ml beaker. Then the mixture is stirred thoroughly. The solution is prepared immediately before usage or at most one day before usage and stored at 4° C.

The preparation for toothpaste supernatant is described. 15 grams of deionized water is added to 5 grams toothpaste in a 100 ml beaker. After stirring thoroughly, the mixture is centrifuge 11,000×g for 20 minutes. The supernatant is prepared immediately before usage or at most one day before usage and stored at 4° C.

After the HA disks are removed from the splint, the disks are used for ex vivo treatment by different sodium bicarbonate solutions and oral care compositions. After being treated with the subject solution/supernatant and labeled with microbial/EPS fluorescent probe and calcium fluorescent probe, the biofilm in the grooves is measured by confocal laser scanning microscopy (CLSM).

Disk preparation is described. The HA disks are rinsed in PBS solution and each HA disk is divided into two halves by tweezers. Thereafter each half-disk specimen is placed into 500-1000 ul of PBS solution statically for 1 minute. Each specimen is treated for two minutes by either PBS solution, sodium bicarbonate solution, or a toothpaste supernatant. Each specimen is washed by holding each disk with tweezers, shaken for ten rounds of back and forth in 1 ml of PBS solution. This washing cycle is repeated. Thereafter each specimen is immersed into 500-1000 ul PBS solution statically for 5 minutes.

Fluorescence staining and microscopy is described. Fluorescence labeled calcium probes are molecules that exhibit an increase in fluorescence upon binding Ca²⁺. Fluo-3™ is used to image the spatial dynamics of Ca²⁺ signaling. Biofilm may be treated with the AM™ ester forms of calcium probes by adding the dissolved probe directly to biofilm. Fluo-3™, AM™, cell permeant fluorescent probes are useful for intracellular and extracellular calcium staining using confocal microscopy, flow cytometry, and microplate screening applications (absorption/emission maxima ˜506/526 nm). It is reported that the Concanavalin ATM (Con A), Alexa Fluor® 594 Conjugate is a reliable alternative to stain EPS of biofilm. Alexa Fluor® 594 conjugate of Con A exhibits the bright, red fluorescence of the Alexa Fluor® 594 dye (absorption/emission maxima ˜590/617 nm). Concanavalin A™, Alexa Fluor® 594 Conjugate selectively binds to α-mannopyranosyl and α-glucopyranosyl residues which are rich in EPS part of biofilm. After treatment and immersing, each half-disk specimen is stained with a dye mixture solution of the Fluo-3™, AM™, cell permeant fluorescent probe together with Concanavalin A™, Alexa Fluor® 594 Conjugate probe (containing 5 uM Fluo-3™+5 uM Con-A™) for 30 minutes in the dark. After staining, each specimen is immersed into 500-1000 ul PBS solution statically for 2 minutes. The specimens are washed again, by holding each disk with tweezers, shaken for five rounds of back and forth in 1 ml PBS solution, and repeated. For Fluo-3/Con-A dye co-stained samples, the following parameters are used: λex=488/561 nm, respectively, λem=526/617 nm respectively, 20× objective lens, and scanning from bottom of disk surface bacteria for 60 um depth with step size of 3 um. Although not shown, the other half-disk can be stained with L7012 LIVE/DEAD® dye solution (containing 5 uM Syto-9+30 uM propidium iodide) for 15 minutes in the dark as a control for assessing bactericidal efficacy. For the L7012 LIVE/DEAD® dyed stained sample, the following parameters are used: λex=488 nm, λem=500/635 nm respectively, 20× objective lens, and scanning from bottom of surface bacteria for 60 um with step size of 3 um.

Confocal Laser Scanning Microscopy (CLSM) is described. The Leica™ TCS SP8 AOBS spectral confocal microscope (available from Leica Mikroskopie GmbH, Wetzlar, Germany) is used. The confocal system consists of a Leica™ DM6000B upright microscope and a Leica™ DMIRE2 inverted microscope. An upright stand is used for applications involving slide-mounted specimens; whereas the inverted stand, having a 37° C. incubation chamber and CO₂ enrichment accessories, provides for live cell applications. The microscopes share an exchangeable laser scan head and, in addition to their own electromotor-driven stages, a galvanometer-driven high precision Z-stage which facilitates rapid imaging in the focal (Z) plane. In addition to epifluorescence, the microscopes support a variety of transmitted light contrast methods including bright field, polarizing light and differential interference contrast, and are equipped with 5×, 20×, 40×, 63× (oil and dry) and 100× (oil) Leica™ objective lenses.

The laser scanning and detection system is described. The TCS SP8 AOBS confocal laser scanning system (available from Leica Lasertechnik GmbH, Heidelberg, Germany) is supplied with four lasers (one diode, one argon, and two helium neon lasers) thus allowing excitation of a broad range of fluorochromes within the UV, visible and far red ranges of the electromagnetic spectrum. The design of the laser scan head, which incorporates acousto-optical tunable filters (AOTF), an acousto-optical beam splitter (AOBS) and four prism spectrophotometer detectors, permits simultaneous excitation and detection of three fluorochromes. The upright microscope also has a transmission light detector making it possible to overlay a transmitted light image upon a fluorescence recording.

Leica™ Confocal software LAS AF3.3.0 is used. The confocal is controlled via a standard Pentium PC equipped with dual monitors and running Leica™ Confocal Software. The Leica Confocal Software LAS AF3.3.0 (available from Leica Lasertechnik GmbH, Heidelberg, Germany) provides an interface for multi-dimensional image series acquisition, processing and analysis, that includes 3D reconstruction and measurement, physiological recording and analysis, time-lapse, fluorochrome co-localization, photo-bleaching techniques such as FRAP and FRET, spectral immixing, and multicolour restoration. Regarding image analysis, Fluo-3™/Con-A™ fluorescence channels are chosen to quantify fluorescence intensity ratio of green pixels (Calcium) to red pixels (EPS) and Con-A™ fluorescence channel is chosen to measure the biofilm thickness.

Turning to Table 1, the fluorescence intensity ratio of Ca/EPS within in situ plaque biofilm and average biofilm thickness are provided for two sodium bicarbonate containing solutions, one commercially available sodium bicarbonate containing toothpaste and a negative control. The procedures previously described are used. The biofilm is treated with the subject oral care compositions first, and then the treated biofilm is labeled with the EPS and calcium probes. Using software, the mean fluorescence intensities of green pixels (staining calcium ions) and red pixels (staining EPS) are given. The fluorescence intensity ratio of green pixels to red pixels is then calculated. Regarding biofilm thickness assessment, six selected fields of Con-A™ fluorescence channel of each specimen are evaluated. These fields are considered as representative of the whole sample after the observer's general examination. The distance is measured from the surface of the biofilm to its base, measuring the thickness of the field, and subsequently the mean thickness of the biofilm of the corresponding specimen is calculated.

In Table 1, SENSODYNE® PARODONTAX™ toothpaste (“PARODONTAX”, LOT#14042610, containing around 67 wt % sodium bicarbonate), an example of commercially available toothpaste composition, is used. 20 wt % sodium bicarbonate solution (“20% BS”) and 60 wt % sodium bicarbonate solution (“60% BS”) are used as positive controls. PBS is used as the negative control. The results indicate that 20% BS shows a significantly lower Ca/EPS ratio than PBS. PARODONTAX and 60% BS show significantly lower Ca/EPS ratio than 20% BS and PBS. There is no significant difference between Ca/EPS ratio of PARODONTAX and 60% BS. The results also indicate 20% BS show a significantly reduced biofilm thickness than PBS. PARODONTAX and 60% BS show significantly reduced biofilm thickness than 20% BS and PBS. There is no significant difference between biofilm thickness of PARODONTAX and 60% BS.

TABLE 1
Fluorescence intensity ratio of Calcium to EPS, and
Average biofilm thickness for sodium bicarbonate containing
solutions/toothpaste and a negative control.

| | Fluorescence intensity | Average biofilm |
|---|---|---|
| | ratio of Calcium to EPS | thickness (um) |
| | (mean ± SD) | (mean ± SD) |
| PBS | 2.35 ± 0.37 | 46.10 ± 1.73 |
| 20% BS | 1.83 ± 0.06 | 27.90 ± 2.02 |
| 60% BS | 1.53 ± 0.18 | 18.47 ± 1.31 |
| PARODONTAX | 1.58 ± 0.02 | 17.37 ± 2.55 |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of quantitating calcium in a biofilm comprising the steps:
(a) optionally treating the biofilm with an oral care composition;
(b) labeling the optionally treated biofilm with a biofilm fluorescent probe;
(c) labeling the optionally treated biofilm with a calcium fluorescent probe; and
(d) quantitating the labeled calcium by measuring fluorescence light emitted from the labeled calcium.

2. The method of claim 1, wherein the method comprises treating the biofilm with the oral care composition for a treatment contact time from 3 seconds to 48 hours.

3. The method of claim 1, wherein the method comprises treating the biofilm with the oral care composition, wherein the oral care composition is selected from the group consisting of dentifrice, toothpaste, leave-on gel, gel, mouthwash, and combinations thereof.

4. The method of claim 1, wherein the biofilm fluorescent probe is selected from a microbial fluorescent probe, an extracellular polymeric substances (“EPS”) fluorescent probe, or combinations thereof.

5. The method of claim 1, wherein the biofilm fluorescent probe is selected from a microbial fluorescent probe, an extracellular polymeric substances (“EPS”) fluorescent probe, or combination thereof; and wherein the step of treating the method comprises treating the biofilm with the oral care composition for a treatment contact time from 1 minute to 3 minutes.

6. The method of claim 1, wherein the biofilm fluorescent probe is a microbial fluorescent probe, and wherein the method further comprises the step of defining a microbial area of the biofilm by measuring fluorescent light emitted from the labeled microbial biofilm.

7. The method of claim 6, wherein the method further comprises the step of quantitating the labeled calcium within the microbial defined area of the biofilm.

8. The method of claim 6, wherein the microbial fluorescent probe comprises a first probe specific for living bacteria and a second probe specific for dead bacteria.

9. The method of claim 1, wherein the biofilm fluorescent probe is an extracellular polymeric substances (“EPS”) fluorescent probe, and wherein the method further comprises the step of defining an EPS area of the biofilm by measuring fluorescent light emitted from the labeled EPS biofilm.

10. The method of claim 9, wherein the method further comprises the step of quantitating the labeled calcium within the EPS defined area of the biofilm.

11. The method of claim 1, wherein the step of quantitating the labeled calcium by measuring fluorescence light emitted from the labeled calcium is by confocal laser scanning microscopy (CLSM).

12. The method of claim 1, wherein the steps of labeling the biofilm and labeling the calcium are before the step of treating the biofilm with the oral care composition.

13. The method of claim 1, wherein the biofilm is an in situ plaque biofilm.

14. The method of claim 1, further comprising the step of having human subjects wear an oral appliance for 6 hours to 4 days; wherein at least a portion of the oral appliance comprises hydroxylapatite (HA) as a surface of the biofilm to adhere to.

15. The method of claim 14, wherein the biofilm is an in situ plaque biofilm.

16. The method of claim 15, wherein the in situ biofilm on the HA surface is treated with the oral care composition either ex vivo or in vivo.

17. The method of claim 6, wherein the microbial fluorescent probe is a fluorescently labeled rRNA-directed oligonucleotide.

18. The method of claim 9, wherein the EPS fluorescent probe is a fluorescently labeled lectin.

19. The method of claim 1, further comprising the step of determining a quantified fluorescence intensity ratio of Calcium to EPS by quantifying the measured fluorescence light from fluorescent labeled calcium ions and measured fluorescent light from EPS of biofilm.

20. The method of claim 2, wherein the oral care composition comprises an effective amount of a calcium ion binder, and wherein the ion fluorescent probe is a calcium fluorescent probe.
